(19) United States
(12) Patent Application Publication
(10) Pub. No.: US 2025/0025493 A1
(43) Pub. Date: Jan. 23, 2025

(54) GLUCOHEXAOSE AND PREPARATION METHOD THEREFOR AND APPLICATION THEREOF, AND HAIR REGENERATION PREPARATION

(71) Applicant: UNIVERSITY OF MACAU, Macau (CN)

(72) Inventors: Chunming WANG, Macau (CN); Xiaoyu YIN, Macau (CN); Lintao WANG, Macau (CN); Zhe ZHANG, Macau (CN)

(21) Appl. No.: 18/708,217

(22) PCT Filed: Feb. 28, 2022
(86) PCT No.: PCT/CN2022/078159

§ 371 Date: May 8, 2024

(30) Foreign Application Priority Data
Nov. 10, 2021 (CN) 202111324427.3

(51) Int. Cl. A61K 31/736; A61P 17/14; C08B 37/00; C12P 19/04; C12P 19/14

(52) U.S. Cl. CPC A61K 31/736; A61P 17/14; C08B 37/0087; C12P 19/04; C12P 19/14

(57) ABSTRACT

The present disclosure relates to the technical field of skin hair regeneration, and in particular to glucohexaose and a preparation method therefor and an application thereof, and a hair regeneration preparation. The molecular structure of the glucohexaose is Man-Man-Man-Man-Glc-Glc. The gulcohexaose can specifically stimulate local macrophages to release a cytokine network mainly comprising CCL5 chemotactic factors, recruit lymphocytes mainly comprising regulatory T cells, induce an immune cascade reaction, regulate an immune microenvironment, activate rapid proliferation and differentiation of hair follicle stem cells at a hair follicle carina and at the lower end of the carina, remarkably promote periodic regeneration of hair follicles, and accelerate the process of driving the hair follicles to enter a growth period from a resting period, so as to achieve the effect of treating alopecia.

### CROSS REFERENCE TO RELATED APPLICATIONS

The present disclosure claims the right of priority for Chinese patent application no. CN 202111324427.3, filed with the China National Intellectual Property Administration on Nov. 10, 2021 and entitled “GLUCOHEXAOSE AND PREPARATION METHOD THEREFOR AND APPLICATION THEREOF, AND HAIR REGENERATION PREPARATION”, which is incorporated herein by reference in its entirety.

### TECHNICAL FIELD

The present disclosure relates to the technical field of skin hair regeneration, and in particular to glucohexaose and a preparation method therefor and an application thereof, and a hair regeneration preparation.

### BACKGROUND

In today's society, with the increasing pressure of people's lives and the gradual increase in attention to appearance, alopecia is becoming more common, which will not only affect the social quality of patients, but may even bring patients mental stress to some extend, which may further develop into a serious mental health problem. Healthy hair not only has important physiological functions for humans, such as protecting the skin from ultraviolet rays, draining sweat and regulating body temperature, but also has important sociological functions. Therefore, researchers have been constantly trying to prevent and treat alopecia in safe and effective ways. However, at present, clinical means for treating alopecia are limited, mainly including drug treatment and surgical treatment. For example, minoxidil (topical drug) and finasteride (oral drug), which have been approved by the FDA, are effective in improving androgenic alopecia to some extent, but there are problems such as low efficacy and recurrence of alopecia after drug withdrawal. In addition, although hair transplant surgery can be used as an alternative treatment method, its invasiveness, limited number of autologous hair follicle donors, low survival rate of transplanted hair follicles and high cost limit its further application. Therefore, there is an urgent need for some safe and effective treatments for alopecia.

### SUMMARY OF THE INVENTION

The present disclosure provides a glucohexaose having a molecular structure of: Man-Man-Man-Man-Glc-Glc.

In an optional embodiment, the glucohexaose contains an acetyl group of natural origin.

In an optional embodiment, the saccharide units of the glucohexaose are linked in a way selected form any one of α-1,4, α-1,3, β-1,4 and β-1,3.

In an optional embodiment, the glucohexaose has a linear structure.

In an optional embodiment, both the glucose unit and the mannose unit are in the D-form.

The present disclosure provides a method for preparing the glucohexaose according to the above-mentioned embodiment, comprising: performing enzyme degradation on glucomannan with cellulose ether to form the glucohexaose.

In an optional embodiment, the glucomannan is a natural polysaccharide. In an optional embodiment, the glucomannan is a Bletillae rhizoma polysaccharide or a konjac polysaccharide.

In an optional embodiment, the enzyme-to-substrate ratio is 1:5-1:20, the enzyme degradation temperature is 35° C.-50° C., and the enzyme degradation time is 24-37 hours.

In an optional embodiment, the method comprises: before performing enzyme degradation, dissolving the raw material glucomannan and then freeze-drying the raw material glucomannan to form a freeze-dried powder, and then performing the enzyme degradation.

In an optional embodiment, post-treatment is performed after the enzyme degradation is completed.

In an optional embodiment, the post-treatment comprises: after the enzyme degradation is completed, heating the reaction system to 80° C.-100° C., and then performing centrifugation to form a supernatant, and then performing column chromatography separation on the supernatant.

The present disclosure provides an application of the glucohexaose according to any one of the above-mentioned embodiments or glucohexaose prepared by the glucohexaose preparation method according to any one of the above-mentioned embodiments in the preparation of a drug for treating alopecia.

In an optional embodiment, the drug is a drug that activates the rapid proliferation and differentiation of hair follicle stem cells at a hair follicle carina and at the lower end of the carina.

In an optional embodiment, the drug is a drug that promotes the periodic regeneration of hair follicles.

In an optional embodiment, the drug is a drug that accelerates the process of driving hair follicles to enter a growth period from a resting period.

In an optional embodiment, the drug is a drug that activates macrophages to release a cytokine network mainly comprising CCL5 chemotactic factors.

In an optional embodiment, the glucohexaose is applied via any one of direct skin application administration, subcutaneous injection administration and microneedle release administration.

The present disclosure provides an application of the glucohexaose according to any one of the above-mentioned embodiments or glucohexaose prepared by the glucohexaose preparation method according to any one of the above-mentioned embodiments in the preparation of a preparation for promoting hair regeneration.

The present disclosure provides a hair regeneration preparation, comprising the glucohexaose according to any one of the above-mentioned embodiments or glucohexaose prepared by the glucohexaose preparation method according to any one of the above-mentioned embodiments.

In an optional embodiment, the hair regeneration preparation further comprises a medically or pharmaceutically acceptable carrier or additive.

In an optional embodiment, the hair regeneration preparation is applied via any one of direct skin application administration, subcutaneous injection administration and microneedle release administration.

The present disclosure provides a method for treating and preventing alopecia, wherein the method comprises:
administering to a subject in need thereof a therapeutically effective amount of the glucohexaose according to any one of the above-mentioned embodiments or glucohexaose prepared by the glucohexaose preparation method according to any one of the above-mentioned embodiments or the hair regeneration preparation.

In an optional embodiment, the mode of administration is selected from any one of direct skin application administration, subcutaneous injection administration and microneedle release administration.

In an optional embodiment, the subject has one of androgenetic alopecia, neurogenic alopecia, endocrine alopecia, nutritional alopecia, generalized alopecia, degenerative alopecia, alopecia areata, trichotillomania, telogen effluvium, anagen effluvium, scarring alopecia, thinning of the scalp, hair shaft disorder, infectious disease, hormonal disorder and drug-induced alopecia.

The present disclosure provides the use of any one of the above-mentioned glucohexaoses or glucohexaose prepared by any one of the above-mentioned glucohexaose preparation methods or the hair regeneration preparation in the treatment of hair regeneration and the prevention of alopecia.

The present disclosure provides the glucohexaose according to any one of the above-mentioned embodiments or a glucohexaose hair regeneration preparation prepared by the glucohexaose preparation method according to any one of the above-mentioned embodiments or the hair regeneration preparation for use in the treatment of hair regeneration and the prevention of alopecia.

### BRIEF DESCRIPTION OF THE DRAWINGS

In order to more clearly illustrate the technical solutions of the examples of the present disclosure, the drawings used in the examples will be briefly introduced below. It should be understood that the following drawings only show certain examples of the present disclosure, and therefore should not be considered as limiting the scope. For a person skilled in the art, other related drawings also can be obtained according to the drawings without creative efforts.

FIG. 1 shows the detection results of experimental example 1 of the present disclosure;

FIG. 2 shows the detection results of experimental example 2 of the present disclosure;

FIG. 3 shows the detection results of experimental example 3 of the present disclosure;

FIG. 4 shows the detection results of experimental example 4 of the present disclosure;

FIG. 5 shows the detection results of experimental example 5 of the present disclosure;

FIG. 6 shows a nuclear magnetic resonance hydrogen spectrum of glucohexaose provided by the examples of the present disclosure.

### DEFINITION OF TERMS

Before the present invention is described by way of embodiments and examples, it should be understood that the terminology used herein is for the purpose of describing particular embodiments and examples only and is not intended to limit the scope of the present invention.

As used herein, in the molecular structure of the glucohexaose, the term “Man” refers to: a mannose unit.

As used herein, in the molecular structure of the glucohexaose, the term “Glc” refers to: a glucose unit.

As used herein, the term “enzyme-to-substrate ratio” refers to the mass ratio of an enzyme to a reaction substrate.

As used herein, the term “hair follicle stem cell” is a hair follicle stem cell in the carina of the outer root sheath of human hair follicles. Hair follicle stem cells are adult stem cells, which are at rest in vivo and exhibit surprising proliferation capacity under in vitro culture conditions.

### DETAILED DESCRIPTION OF EMBODIMENTS

In order to make the objectives, technical solutions and advantages of examples of the present disclosure clearer, the technical solutions in the examples of the present disclosure will be clearly and completely described below. If specific conditions are not specified in the examples, conventional conditions or conditions recommended by a manufacturer are followed. The reagents or instruments used therein for which manufacturers are not specified are all conventional products that are commercially available.

An embodiment of the present disclosure provides a glucohexaose, wherein the glucohexaose has six saccharide units, comprises glucose units and mannose units, and has a molecular structure of: Man-Man-Man-Man-Glc-Glc. The gulcohexaose having the structure can specifically stimulate macrophages and mediate an immune response, so that local macrophages of skin release a cytokine network mainly comprising CCL5 chemotactic factors, and on this basis, recruit lymphocytes mainly comprising regulatory T cells, induce an immune cascade reaction, regulate an immune microenvironment, activate rapid proliferation and differentiation of hair follicle stem cells at a hair follicle carina and at the lower end of the carina, remarkably promote periodic regeneration of hair follicles, and accelerate the process of driving the hair follicles to enter a growth period from a resting period, so as to achieve the effect of treating alopecia. This provides new ideas for the development of novel, effective and safe hair regeneration preparations and alopecia treatment approaches.

In some embodiments, the gulcohexaose contains an acetyl group of natural origin or does not contain an acetyl group of natural origin, and the number of acetyl groups contained therein also varies, such as 0, 1 or 2, depending on the origin of the gulcohexaose. It is believed that without being bound by theory, the acetyl group is randomly linked at the C-6 position of the mannose unit in the gulcohexaose structure, the C-6 position is —OH in the absence of the acetyl group, and the C-6 position is —OCOCH₃ in the presence of the acetyl group. In some embodiments, the saccharide units of the glucohexaose are linked in a way selected form any one of α-1,4, α-1,3, β-1,4 and β-1,3. The glucohexaose has a linear structure. In an optional embodiment, both the glucose unit (Glc) and the mannose unit (Man) are in the D-form. An embodiment of the present disclosure provides a method for preparing the glucohexaose, wherein the method comprises:
dissolving a raw material glucomannan in water, and then freeze-drying the formed solution of the raw material glucomannan to form a freeze-dried powder;
and then performing enzyme degradation on the glucomannan, i.e., the above-mentioned freeze-dried powder, with a cellulase. In some embodiments, the above-mentioned freeze-dried powder and cellulase are fully dissolved in water, wherein the enzyme-to-substrate ratio is 1:5-1:20, such as 1:5-1:10, 3:5-5:20 or 1:5-5:15. For example, the enzyme-to-substrate ratio is any value between 1:5-1:20, such as 1:5, 1:6, 1:7, 1:8, 1:9, 1:10, 1:11, 1:12, 1:13, 1:14, 1:15, 1:16, 1:17, 1:18, 1:19 and 1:20. The enzyme degradation temperature is 35° C.-50° C., such as 35° C.-40° C., 40° C.-50° C. or 35° C.-45° C. For example, the temperature is any value between 35° C.-50° C., such as 35° C., 36° C., 37° C., 38° C., 39° C., 40° C., 41° C., 42° C., 43° C., 44° C., 45° C., 46° C., 47° C., 48° C., 49° C. and 50° C. The enzyme degradation time is 24-37 hours, such as 25-37 hours, 25-35 hours, or 25-30 hours. For example, the enzyme degradation time is any value between 24-37 hours, such as 24 hours, 25 hours, 26 hours, 27 hours, 28 hours, 29 hours, 30 hours, 31 hours, 32 hours, 33 hours, 34 hours, 35 hours, 36 hours and 37 hours. After the enzyme degradation is completed, the reaction system is heated to 80° C.-100° C., such as 80° C.-90° C., 90° C.-100° C. or 85° C.-95° C., for example, any value between 80° C.-100° C., such as 80° C., 82° C., 85° C., 86° C., 89° C., 90° C., 91° C., 93° C., 95° C., 97° C., 99° C. and 100° C. Accordingly, the cellulase is inactivated, then centrifugation is performed to form a supernatant, and then column chromatography separation is performed on the supernatant.

In some embodiments, the glucomannan is a natural polysaccharide, such as a Bletillae rhizoma polysaccharide or a konjac polysaccharide; and the Bletillae rhizoma polysaccharide and the konjac polysaccharide are polysaccharides extracted from the rhizomes of Bletillae rhizoma or konjac plants respectively.

An embodiment of the present disclosure provides an application of the above-mentioned glucohexaose or the glucohexaose prepared by the above-mentioned glucohexaose preparation method in the preparation of a drug for treating alopecia. The drug can be a drug that activates the rapid proliferation and differentiation of hair follicle stem cells at a hair follicle carina and at the lower end of the carina; can also be a drug that promotes the periodic regeneration of hair follicles; or a drug that accelerates the process of driving hair follicles to enter a growth period from a resting period; or a drug that activates macrophages to release a cytokine network mainly comprising CCL5 chemotactic factors; can also be a drug that achieves the above-mentioned efficacy at the same time.

The glucohexaose used in the present disclosure can be used to treat androgenetic alopecia in mouse models via any one of direct skin application administration, subcutaneous injection administration and microneedle release administration, and the hair formation is observed. It is found that using the glucohexaose for alopecia treatment can effectively promote the growth of hair on the back of mice without any serious adverse reactions, indicating that it is a safe and effective treatment method and provides a new idea for treating hair growth disorders.

An embodiment of the present disclosure further provides an application of the above-mentioned glucohexaose or the glucohexaose prepared by the above-mentioned glucohexaose preparation method in the preparation of a preparation for promoting hair regeneration.

An embodiment of the present disclosure further provides a hair regeneration preparation, which comprises the above-mentioned glucohexaose or the glucohexaose prepared by the above-mentioned glucohexaose preparation method, and further comprises a medically or pharmaceutically acceptable carrier or additive; and the hair regeneration preparation is applied via any one of direct skin application administration, subcutaneous injection administration and microneedle release administration.

An embodiment of the present disclosure further provides a method for treating and preventing alopecia, wherein the method comprises:
administering to a subject in need thereof a therapeutically effective amount of the glucohexaose according to any one of the above-mentioned embodiments or glucohexaose prepared by the glucohexaose preparation method according to any one of the above-mentioned embodiments or the hair regeneration preparation.

In some embodiments, the mode of administration is selected from any one of direct skin application administration, subcutaneous injection administration and microneedle release administration.

In some embodiments, the subject has one of androgenetic alopecia, neurogenic alopecia, endocrine alopecia, nutritional alopecia, generalized alopecia, degenerative alopecia, alopecia areata, trichotillomania, telogen effluvium, anagen effluvium, scarring alopecia, thinning of the scalp, hair shaft disorder, infectious disease, hormonal disorder and drug-induced alopecia.

An embodiment of the present disclosure further provides the use of the glucohexaose according to any one of the above-mentioned embodiments or glucohexaose prepared by the glucohexaose preparation method according to any one of the above-mentioned embodiments or the hair regeneration preparation in the treatment of hair regeneration and the prevention of alopecia.

An embodiment of the present disclosure further provides the glucohexaose according to any one of the above-mentioned embodiments or a glucohexaose hair regeneration preparation prepared by the glucohexaose preparation method according to any one of the above-mentioned embodiments or the hair regeneration preparation for use in the treatment of hair regeneration and the prevention of alopecia.

The gulcohexaose provided by the present disclosure can effectively activate rapid proliferation and differentiation of hair follicle stem cells at a hair follicle carina and at the lower end of the carina, remarkably promote periodic regeneration of hair follicles, and accelerate the process of driving the hair follicles to enter a growth period from a resting period, so as to achieve the effect of treating alopecia.

The gulcohexaose provided by the embodiments of the present disclosure can specifically stimulate local macrophages to release a cytokine network mainly comprising CCL5 chemotactic factors, recruit lymphocytes mainly comprising regulatory T cells, induce an immune cascade reaction, regulate an immune microenvironment, activate rapid proliferation and differentiation of hair follicle stem cells at a hair follicle carina and at the lower end of the carina, remarkably promote periodic regeneration of hair follicles, and accelerate the process of driving the hair follicles to enter a growth period from a resting period, so as to achieve the effect of treating alopecia.

The characteristics and performance of the present disclosure will be described in detail below in combination with the examples.

### EXAMPLES

### Example 1 Preparation of Gulcohexaose

The example of the present disclosure provides a method for preparing a glucohexaose, wherein the method comprises the following steps:

5 g of konjac powder extracted from the tuber part of a natural plant konjac was weighed and completely dissolved in 100 ml of deionized water, the resulting solution was fully stirred for 24 h, and a konjac polysaccharide solution was then subjected to freeze-drying treatment to obtain a freeze-dried konjac glucomannan powder. The method for preparing the konjac polysaccharide is as follows: the rhizome part of natural konjac was taken and crushed by a traditional Chinese medicine crusher to obtain a konjac powder; and the konjac powder was repeatedly washed with an aqueous ethanol solution to obtain the konjac polysaccharide after crude extraction.

Next, the freeze-dried konjac polysaccharide powder was subjected to cellulase (purchased from: Shanghai Aladdin Biochemical Technology Co., Ltd. (Shanghai, China)) degradation treatment. First, 500 mg of the freeze-dried konjac glucomannan powder was weighed and fully dissolved in 100 ml of deionized water, with a reaction temperature of 40° C. and a reaction time of 24 h; after the reaction was completed, the reaction system was heated to 100° C. to inactivate the cellulase, after the reaction was terminated, filtration and centrifugation were performed to remove the cellulase and the incompletely reacted glucomannan, and the supernatant was retained, which was the oligosaccharide solution; and the oligosaccharide solution was subjected to separation and purification to obtain the target product of gulcohexaose, the target product was subjected to separation and purification by Sephadex G-25 dextran chromatography, the eluates were collected and separated in fractions, and freeze-drying was performed to obtain gulcohexaose with a purity of 95% or more. The column chromatography separation conditions are as follows: at room temperature, the mobile phase is 100% deionized water. During the elution, the collected fractions were identified by thin layer chromatography (TLC) to determine the collected products.

The gulcohexaose prepared by the above preparation method was characterized (see FIG. 6), and its molecular structure is as follow: Man-Man-Man-Man-Glc-Glc.

### Example 2 Preparation of Gulcohexaose

The preparation method of example 2 is similar to that of example 1, except that the source of the raw material is different. In example 2, the dried tuber of the Chinese medicinal material Bletillae rhizoma is selected as the raw material, and the other unmentioned aspects of example 2 are the same as those in the preparation method of example 1.

Example 3 Preparation of Gulcohexaose

The method of example 3 is similar to that of example 1, except that the enzyme used for the enzyme degradation treatment is different. In this example, endo-1,4-β-mannanase (purchased from: Megazyme Ltd.) is selected for the enzyme degradation of the freeze-dried konjac polysaccharide powder, and the other undescribed aspects of example 3 are the same as those in the preparation method of example 1.

### Experimental Example 1

### Induction and Activation Experiment

The gulcohexaose sample prepared in example 1 above was used to perform an induction and activation experiment on mouse bone marrow-derived macrophages (mBMDM, primary cells extracted from C57BL/6J mice by laboratory methods):

Mouse bone marrow-derived monocytes (5×10⁶ cells/10 cm petri dish) were seeded in 10 cm petri dishes and cultured using L929 cell culture supernatant obtained after 7 days of culture+RPMI-1640 medium+10% fetal bovine serum+1% penicillin-streptomycin in a 37° C., 5% CO₂ incubator. After cell adherence, the cells were co-incubated with 50 ug/ml gulcohexaose for 24 h, designated as OG6 group, while a blank group (an experimental group to which the gulcohexaose was not added and to which an equal volume (10 μl) of PBS buffer (mainly consisted of Na₂HPO₄, KH₂PO₄, NaCl and KCl) was added) was set up, designated as PBS group. After 24 h of co-incubation, the total RNA was extracted from the sample using TRIzol (Invitrogen), eukaryotic mRNA sequencing experiment analysis and bioinformatics analysis were performed, and the difference between the expression level of cytokines released by the mouse bone marrow-derived macrophages stimulated by the gulcohexaose and the expression level of cytokines released in the blank group was compared. This experiment was repeated twice.

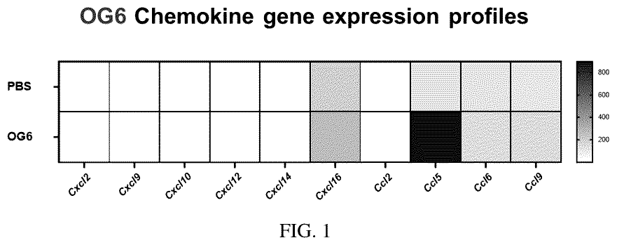

See FIG. 1 for the results. It can be seen from FIG. 1 that the gulcohexaose can specifically activate macrophages to release a cytokine network mainly comprising CCL5 chemotactic factors.

### Experimental Example 2

### Verification Experiment

To verify the experimental results of eukaryotic mRNA sequencing analysis, a real-time fluorescence quantitative PCR experiment was used to determine the expression level of Ccl5 gene in macrophages stimulated by the gulcohexaose. The gulcohexaose prepared in example I was used to perform an induction and activation experiment on mouse bone marrow-derived macrophages (mBMDM, primary cells extracted from C57BL/6J mice by laboratory methods):
mouse bone marrow-derived monocytes (5×10⁶ cells/10 cm petri dish) were seeded in 10 cm petri dishes and cultured using L929 cell culture supernatant obtained after 7 days of culture+RPMI-1640 medium+10% fetal bovine serum+1% penicillin-streptomycin in a 37° C., 5% CO₂ incubator. After cell adherence, the cells were co-incubated with 50 ug/ml gulcohexaose for 1 h, 12 h and 24 h, sequentially designated as 1 hr group, 12 hr group and 24 hr group, while a blank group (an experimental group to which the gulcohexaose was not added and to which an equal volume (10 μl) of PBS buffer (the components thereof being the same as those in experimental example 1) was added) was set up, designated as PBS group. Three groups of parallel experiments were set up respectively. After co-incubation for different time periods, the total RNA was extracted from the samples respectively, and the expression levels of Ccl5 cytokine in mouse bone marrow-derived macrophages before and after the stimulation by the gulcohexaose were detected by real-time quantitative PCR.

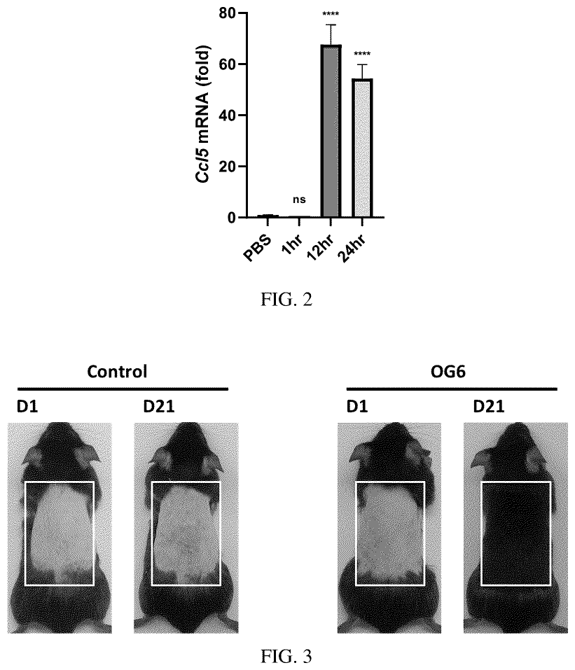

See FIG. 2 for the results. It can be seen from FIG. 2 that the gulcohexaose can specifically stimulate macrophages to rapidly upregulate the expression level of Ccl5 cytokine, with a significant change after 12 hours of stimulation and a high expression level of Ccl5 cytokine in macrophages maintained even after 24 hours of stimulation.

### Experimental Example 3

### Animal Experiment-Direct Application

The gulcohexaose sample prepared in example 1 was investigated for the biological effect in promoting hair follicle regeneration.

The method is as follows: healthy male 7-week-old C57BL/6J mice were subjected

to back unhairing treatment, wherein the hair on the back area was completely removed using an unhairing cream (Veet); starting from D-0, testosterone was topically applied to the back unhairing area of the mice every day to establish a mouse model of androgenetic alopecia (AGA), and the hair growth of the mice was photographed for recording; the gulcohexaose was directly and topically applied to the back unhairing area of the mice every seven days (D-0, D-7, D-14), designated as OG6 group; a blank group (no applying) was set up, designated as a control group; and the hair growth on the back of the mice was observed on day 21.

See FIG. 3 for the results. It can be seen from FIG. 3 that the hair follicles in the back treatment area of the mice in the experimental group with the direct application of the gulcohexaose for alopecia treatment have entered a growth period from a resting period and grew hair compared with those in the blank group. This indicates that the gulcohexaose absorbed through the skin specifically stimulates local macrophages of the skin tissue to release a cytokine network mainly comprising CCL5, regulates the local immune microenvironment, and promotes the proliferation of hair follicle stem cells, thereby accelerating the transition of hair follicles in the unhairing treatment area of the mice from a resting period to a growth period. On day 21, the hair growth at the part treated with the direct application of the gulcohexaose is obviously superior to that on the mice in the blank group.

### Experimental Example 4

### Animal Experiment-Subcutaneous Injection

The method is as follows: healthy male 7-week-old C57BL/6J mice were subjected to back unhairing treatment, wherein the hair on the back area was completely removed using an unhairing cream (Veet); starting from D-0, testosterone was topically applied to the back unhairing area of the mice every day to simulate androgenetic alopecia (AGA), and the hair growth of the mice was photographed for recording; the gulcohexaose was subcutaneously injected to the back unhairing area of the mice every seven days (D-0, D-7, D-14), designated as OG6 group; a blank group (no applying) was set up, designated as a control group; and the hair growth on the back of the mice was observed on day 21.

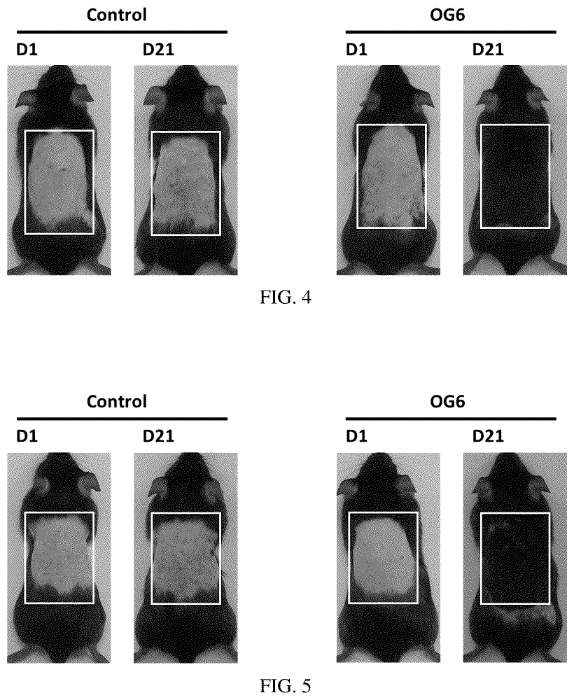

See FIG. 4 for the results. It can be seen from FIG. 4 that the hair follicles in the back treatment area of the mice in the experimental group with the subcutaneous injection of the gulcohexaose have entered a growth period from a resting period and grew hair compared with those in the blank group. This indicates that the gulcohexaose injected subcutaneously can specifically stimulate local macrophages of the skin tissue to release a cytokine network mainly comprising CCL5, regulate the local immune microenvironment, and promote the proliferation of hair follicle stem cells, thereby obviously inducing the hair follicles in the back unhairing area of the mice to enter a regeneration period. On day 21, the hair growth of the mice in the experimental group treated with the subcutaneous injection of the gulcohexaose is obviously superior to that on the mice in the blank group.

### Experimental Example 5

### Animal Experiment-Microneedle Administration

The method is as follows: healthy male 7-week-old C57BL/6J mice were subjected to back unhairing treatment, wherein the hair on the back area was completely removed using an unhairing cream (Veet); starting from D-0, testosterone was topically applied to the back unhairing area of the mice every day to simulate androgenetic alopecia (AGA) for modeling, and the hair growth of the mice was photographed for recording; the gulcohexaose was microneedle administered to the back unhairing area of the mice every seven days (D-0, D-7, D-14), designated as OG6 group; a blank group (no applying) was set up, designated as a control group; and the hair growth on the back of the mice was observed on day 21.

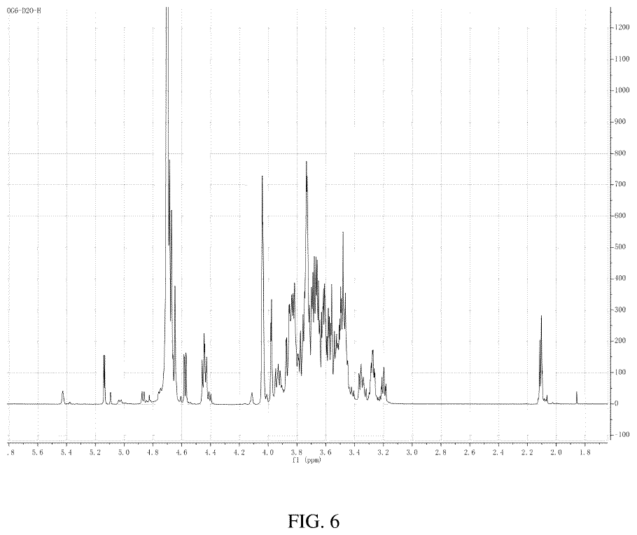

See FIG. 5 for the results. It can be seen from FIG. 5 that the hair follicles in the back treatment area of the mice in the experimental group with the microneedle administration of the gulcohexaose for treatment are the first to enter a growth period from a resting period and are the first to grow hair compared with those in the blank group. The gulcohexaose promotes the proliferation of epidermal stem cells and hair follicle stem cells, and remarkably promotes the transformation of the hair follicles from a late resting period to a growth period in the mice. The hair growth at the part treated with the gulcohexaose is obviously denser than that on the mice in the blank group.

For the treatment of alopecia via the microneedle administration of the gulcohexaose, not only the skin can be activated to initiate a repair response through appropriate trauma so as to avoid the possibility of scarring and hair follicle structure damage caused by massive trauma during clinical application, but also the gulcohexaose can be put into full play in stimulating and inducing macrophages to release a cytokine network mainly comprising CCL5 so as to regulate an immune microenvironment, which, on this basis, activates hair follicle stem cells to proliferate so as to promote hair follicle regeneration, thereby improving alopecia.

The above is only optional examples of the present disclosure, and is not intended to limit the present disclosure. For a person skilled in the art, the present disclosure may have various changes and variations. Any modification, equivalent substitution, improvement, etc. made within the spirit and principle of the present disclosure shall fall within the scope of protection of the present disclosure.

### INDUSTRIAL APPLICABILITY

The gulcohexaose provided by the present disclosure can effectively activate rapid proliferation and differentiation of hair follicle stem cells at a hair follicle carina and at the lower end of the carina, remarkably promote periodic regeneration of hair follicles, and accelerate the process of driving the hair follicles to enter a growth period from a resting period, so as to achieve the effect of treating alopecia, having wide applicability and excellent market value.

1-15. (canceled)

16. A glucohexaose, wherein the glucohexaose has a molecular structure of: Man-Man-Man-Man-Glc-Glc.

17. The glucohexaose according to claim 16, wherein the glucohexaose contains an acetyl group of natural origin.

18. The glucohexaose according to claim 16, wherein the saccharide units of the glucohexaose are linked in a way selected form any one of α-1,4, α-1,3,-1,4 and β-1,3.

19. The glucohexaose according to claim 18, wherein the glucohexaose has a linear structure.

20. The glucohexaose according to claim 18, wherein both the glucose unit and the mannose unit are in D-form.

21. A method for preparing the glucohexaose according to claim 16, wherein the method comprises a step of: performing enzyme degradation on glucomannan with cellulose ether to form the glucohexaose.

22. The preparation method according to claim 21, wherein the glucomannan is a natural polysaccharide.

23. The preparation method according to claim 22, wherein the natural polysaccharide is a Bletillae rhizoma polysaccharide or a konjac polysaccharide.

24. The preparation method according to claim 22, wherein enzyme-to-substrate ratio is 1:5-1:20, enzyme degradation temperature is 35° C.-50° C., and enzyme degradation time is 24-37 hours.

25. The preparation method according to claim 21, wherein the method comprises a step of: before performing enzyme degradation, dissolving the raw material glucomannan and then freeze-drying the raw material glucomannan to form a freeze-dried powder, and then performing the enzyme degradation.

26. The preparation method according to claim 25, wherein the method further comprises a post-treatment step performed after the enzyme degradation is completed.

27. The preparation method according to claim 26, wherein the post-treatment step comprises: after the enzyme degradation is completed, heating the reaction system to 80° C.-100° C., and then performing centrifugation to form a supernatant, and then performing column chromatography separation on the supernatant.

28. A hair regeneration preparation, wherein the hair regeneration preparation comprises the glucohexaose according to claim 16.

29. The hair regeneration preparation according to claim 28, wherein the hair regeneration preparation further comprises a medically or pharmaceutically acceptable carrier or additive.

30. The hair regeneration preparation according to claim 28, wherein the hair regeneration preparation is applied via any one of: direct skin application administration, subcutaneous injection administration and microneedle release administration.

31. A hair regeneration preparation, wherein the hair regeneration preparation comprises the glucohexaose prepared by the glucohexaose preparation method according to claim 21.

32. The hair regeneration preparation according to claim 31, wherein the hair regeneration preparation further comprises a medically or pharmaceutically acceptable carrier or additive.

33. The hair regeneration preparation according to claim 31, wherein the hair regeneration preparation is applied via any one of: direct skin application administration, subcutaneous injection administration and microneedle release administration.

34. A method for treating and preventing alopecia, wherein the method comprises a step of: administering to a subject in need thereof a therapeutically effective amount of the glucohexaose according to claim 16.

35. The method according to claim 34, wherein
mode of the administration is selected from any one of: direct skin application administration, subcutaneous injection administration and microneedle release administration.

36. The method according to claim 34, wherein
the subject has one of the following: androgenetic alopecia, neurogenic alopecia, endocrine alopecia, nutritional alopecia, generalized alopecia, degenerative alopecia, alopecia areata, trichotillomania, telogen effluvium, anagen effluvium, scarring alopecia, thinning of the scalp, hair shaft disorder, infectious disease, hormonal disorder and drug-induced alopecia.

37. A method for treating and preventing alopecia, wherein the method comprises a step of:
administering to a subject in need thereof a therapeutically effective amount of the glucohexaose prepared by the glucohexaose preparation method according to claim 21.

38. The method according to claim 37, wherein
mode of the administration is selected from any one of: direct skin application administration, subcutaneous injection administration and microneedle release administration.

39. The method according to claim 37, wherein
the subject has one of the following: androgenetic alopecia, neurogenic alopecia, endocrine alopecia, nutritional alopecia, generalized alopecia, degenerative alopecia, alopecia areata, trichotillomania, telogen effluvium, anagen effluvium, scarring alopecia, thinning of the scalp, hair shaft disorder, infectious disease, hormonal disorder and drug-induced alopecia.
